(19) United States
(12) Patent Application Publication
(10) Pub. No.: US 2012/0142908 A1
(43) Pub. Date: Jun. 7, 2012

(54) COMPOUNDS FOR THE SYNTHETIC INTRODUCTION OF N-ALKYL NUCLEOSIDES INTO DNA OLIGONUCLEOTIDES

(75) Inventors: David A. Berry, Ann Arbor, MI (US); Lana L. Berry, Ann Arbor, MI (US); John C. Hodges, Ann Arbor, MI (US)

(73) Assignee: Berry and Associates, Inc., Dexter, MI (US)

(21) Appl. No.: 13/306,430

(22) Filed: Nov. 29, 2011

Related U.S. Application Data
(60) Provisional application No. 61/418,685, filed on Dec. 1, 2010.

(51) Int. Cl. C07H 1/00; C07H 19/173; C07H 19/073

(52) U.S. Cl. 536/26.71; 536/26.8; 536/26.7

(57) ABSTRACT

The present invention provides for compounds of Formula I:

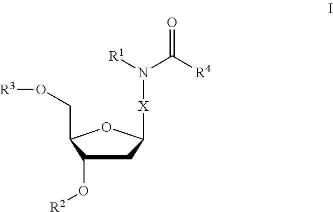

wherein X—N and R¹-R⁴ have any of the values disclosed in the specification. The compounds of Formula I are useful as reagents to introduce N-alkyl nucleosides into DNA oligonucleotides. The present invention also provides for methods of synthesizing the compounds of Formula I.

### CROSS-REFERENCE TO RELATED APPLICATIONS

This application claims the benefit of U.S. Provisional Application No. 61/418,685, filed Dec. 1, 2010, entitled “COMPOUNDS FOR THE SYNTHETIC INTRODUCTION OF N-ALKYL NUCLEOSIDES INTO DNA OLIGONUCLEOTIDES.”

### FIELD OF THE INVENTION

The present invention relates generally to deoxynucleoside derivatives that are useful for the introduction of N-alkyl deoxynucleosides into DNA oligonucleotides. In particular, this invention pertains to N-acyl-N-alkyl deoxynucleoside derivatives. The N-acyl protecting group prevents the undesired formation of branched oligonucleotides during automated or manual DNA synthesis. Removal of the N-acyl protecting group after DNA synthesis affords a DNA oligonucleotide including an N-alkyl deoxynucleoside.

### BACKGROUND

As synthesized in nature, DNA is exists as a polymer comprising a combination of four 2′-deoxynucleosides: 2′-deoxyadenosine (“dA”), 2′-deoxycytidine (“dC”), 2′-deoxyguanosine (“dG”), and 2′-deoxythymidine (“dT”). Each of dA, dC, and dG contains a heterocyclic nucleobase bearing an exocyclic amino group. Alkylation of these exocyclic amino groups occurs sporadically in nature. The resulting alkylated deoxynucleosides disrupt the normal function of DNA. They are involved in carcinogenesis, apoptosis, toxic inhibition of RNA production, and other disruptions of normal cellular function. They are also believed to be involved in DNA repair mechanisms.

In view of the disruptive effect of N-alkyl deoxynucleosides on DNA function, scientific investigation of N-alkylated DNA would be useful in understanding the molecular mechanisms of certain diseases. The synthetic introduction of N-alkyl deoxynucleosides at specific locations of DNA fragments is essential to such investigation.

Traditionally, two synthetic approaches have been employed to introduce N-alkyl deoxynucleosides into synthetic DNA fragments. The first synthetic approach involves the use of a convertible 2′-deoxynucleoside phosporamidite. For example, 4-triazolyl-dU CEP may be employed in place of dC CEP during automated DNA synthesis (Background Scheme 1). In a post-synthetic modification, displacement of the 4-triazolyl group with a primary alkyl amine affords an oligonucleotide having an N(4)-alkyl-dC residue at the desired sequence position.

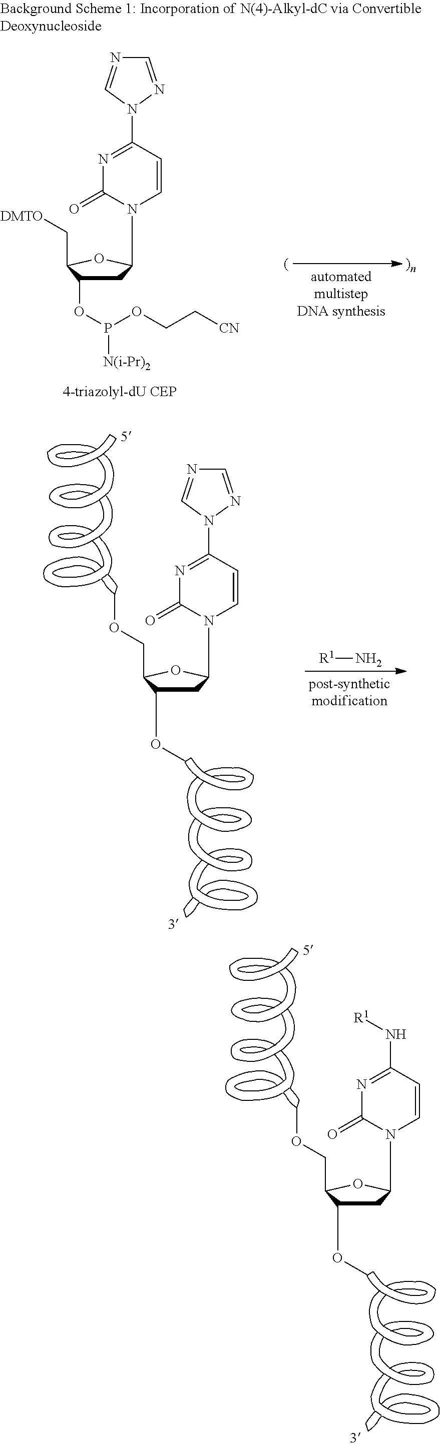

Similarly, O(6)-phenyl-dI CEP may be employed in place of dA CEP during automated DNA synthesis (Background Scheme 2). Displacement of the 6-phenoxy group with a primary alkyl amine affords an oligonucleotide having an N(6)-alkyl-dA residue at the desired sequence position.

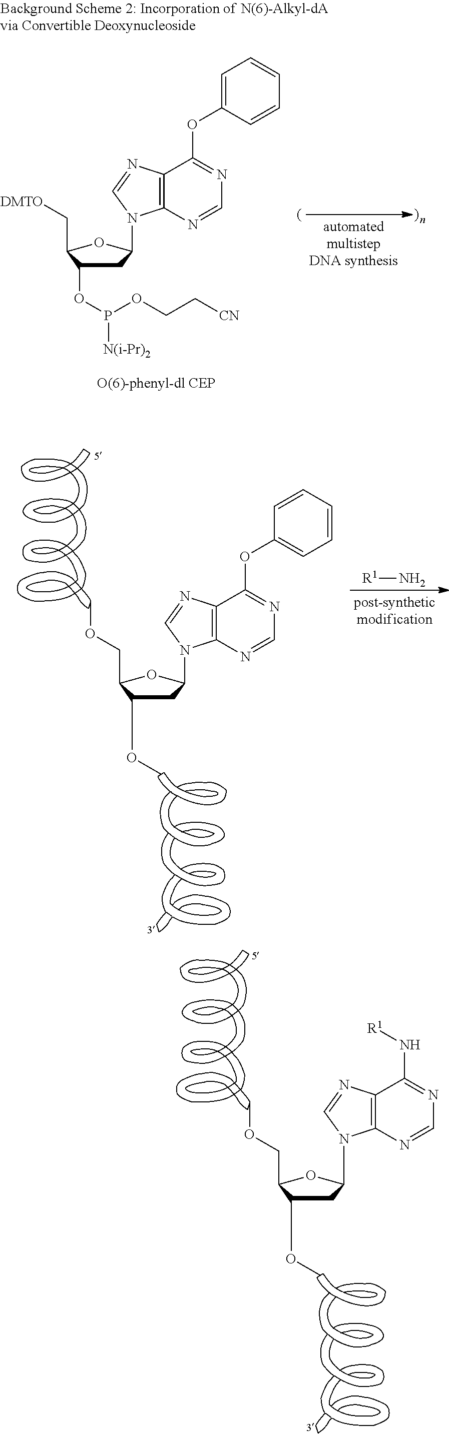

In addition, 2-fluoro-dI CEP may be employed in place of dG CEP during automated DNA synthesis (Background Scheme 3). Displacement of the fluoride substituent with a primary alkyl amine affords an oligonucleotide having an N(2)-alkyl-dG residue at the desired sequence position.

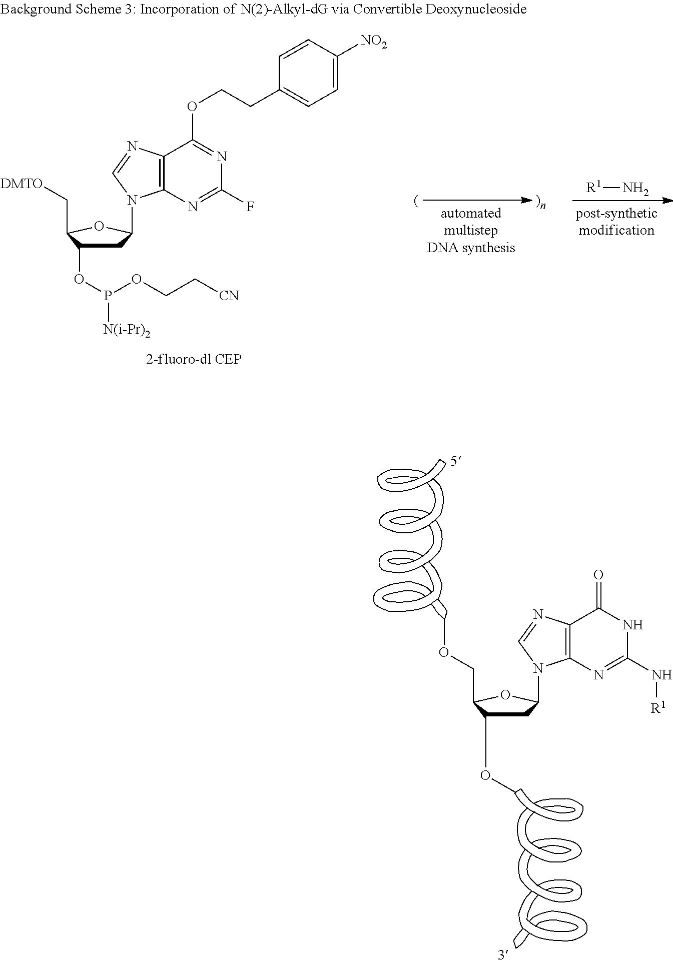

Convertible nucleoside approaches, although useful, have several disadvantages. First, the nucleophilic aromatic substitution reactions used to install the alkyl amine functionality typically require harsh reaction conditions. These reaction conditions promote various side reactions, which create undesirable oligonucleotide contaminants that are difficult to separate from the desired oligonucleotides. Second, the post-synthetic modification typically requires manual intervention at the end of automated DNA synthesis since DNA synthesizers are not equipped to effect such modifications.

The second synthetic approach to introducing an N-alkyl deoxynucleoside into a synthetic DNA fragment involves the use of an N-alkyl-2′-deoxynucleoside phosphoramidite. For example, the use of N(4)-alkyl-dC CEP in place of dC CEP during automated DNA synthesis affords an oligonucleotide having an N(4)-alkyl-dC residue at the desired sequence position (Background Scheme 4). Similarly, the use of N(6)-alkyl-dA CEP or N(2)-alkyl-dG CEP in place of dA CEP or dG CEP, respectively, affords an oligonucleotide having an N(6)-alkyl-dA residue or an N(2)-alkyl-dG residue, respectively, at the desired sequence position. This strategy has the advantage that the oligonucleotide containing the N-alkyl deoxynucleoside is obtained without the need for a manual post-synthetic modification. Unfortunately, this approach is also prone to a particular side reaction in which DNA synthesis occurs on the amine of the deoxynucleoside base. This side reaction produces an undesired branched oligonucleotide.

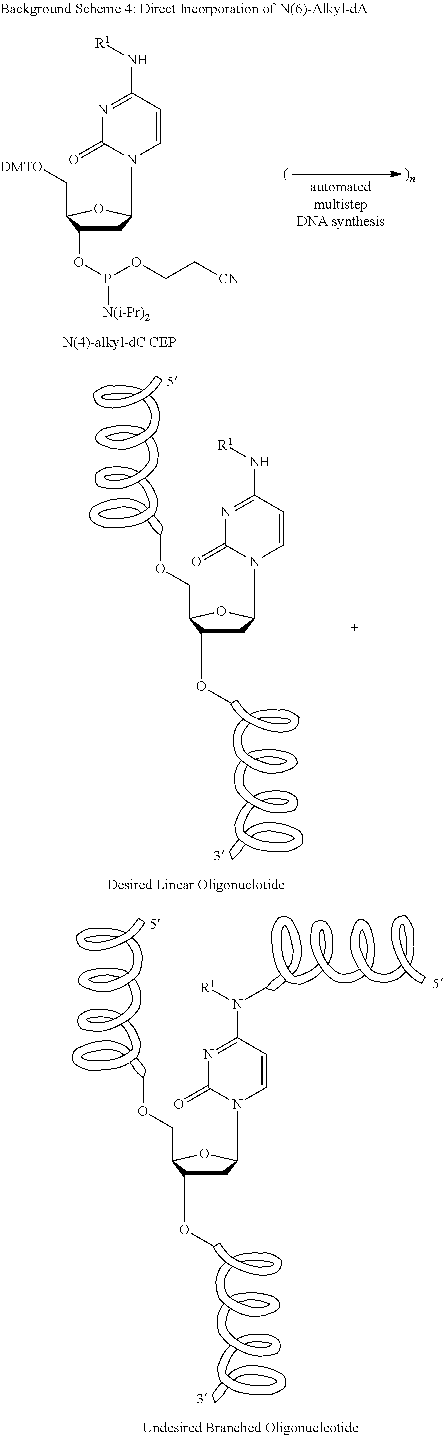

In view of the disadvantages inherent in both of the traditional approaches discussed above, there is a need in the art for strategies to introduce N-alkyl deoxynucleosides into DNA oligonucleotides.

N-Protection strategies for the exocyclic amino substituents of dC CEP, dA CEP, and dG CEP are well known to those skilled in the art of automated DNA synthesis. In contrast, N-protection strategies for N-alkyl deoxynucleoside CEPs are rare. U.S. Pat. No. 6,001,611 (the '611 patent) discloses several N-benzoyl-N-benzyl deoxynucleosides, which are suitable for the introduction of N-benzyl deoxynucleosides into DNA oligonucleotides. However, the '611 patent does not disclose any N-alkanoyl-N-alkyl deoxynucleosides suitable for the introduction of N-(n-alkyl) deoxynucleosides into DNA oligonucleotides.

### SUMMARY OF THE INVENTION

In one embodiment, the present invention provides for compounds of Formula I:

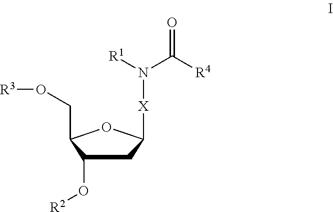

wherein X—N is a heterocyclic nucleobase and its exocyclic amine substituent; R¹ is C₁-C₁₂ n-alkyl; R² and R³ are each independently CEP or DMT; and R⁴ is CH₃, C₂H₅, CF₃, or CCl₃; with the proviso that R² and R³ are different.

In a second embodiment, the present invention provides a process for preparing a compound of Formula II:

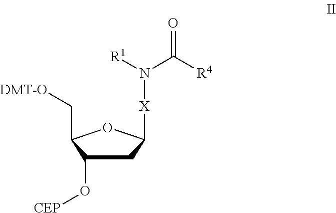

wherein X—N is a heterocyclic nucleobase and its exocyclic amine substituent; R¹ is C₁-C₁₂ n-alkyl; and R⁴ is CH₃, C₂H₅, CF₃, or CCl₃. The process involves the steps of
(a) treating an N-alkyl deoxynucleoside of the formula

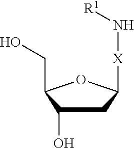

wherein X—N and R¹ are defined as above with a suitable dimethoxytritylating agent to afford an N-alkyl-O(5′)-DMT deoxynucleoside;
(b) treating the N-alkyl-O(5′)-DMT deoxynucleoside with a suitable acylating agent to afford an N-acyl-N-alkyl-O(5′)-DMT deoxynucleoside; and
(c) treating the N-acyl-N-alkyl-O(5′)-DMT deoxynucleoside with a suitable 2-cyanoethyl-N,N-diisopropylaminophosphitylating agent to afford a compound of Formula II.

In a third embodiment, the present invention provides a process for preparing a compound of Formula II:

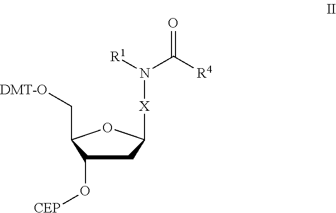

wherein X—N is a heterocyclic nucleobase and its exocyclic amine substituent; R¹ is C₁-C₁₂ n-alkyl; and R⁴ is CH₃, C₂H₅, CF₃, or CCl₃. The process involves the steps of:
(a) treating a deoxynucleoside with a suitable silylating agent and a suitable base to afford an O(3′)-SPG-O(5′)-SPG deoxynucleoside;
(b) treating the O(3′)-SPG-O(5′)-SPG deoxynucleoside with a suitable acylating agent to afford an N-acyl-O(3′)-SPG-O(5′)-SPG deoxynucleoside;
(c) treating the N-acyl-O(3′)-SPG-O(5′)-SPG deoxynucleoside with a suitable alkylating agent to afford an N-acyl-N-alkyl-O(3′)-SPG-O(5′)-SPG deoxynucleoside;
(d) desilylating the N-acyl-N-alkyl-O(3′)-SPG-O(5′)-SPG deoxynucleoside by treating the N-acyl-N-alkyl-O(3′)-SPG-O(5′)-SPG deoxynucleoside with a suitable desilylating agent to afford an N-acyl-N-alkyl deoxynucleoside;
(e) treating the N-acyl-N-alkyl deoxynucleoside with a suitable dimethoxytritylating agent to afford an N-acyl-N-alkyl-O(5′)-DMT deoxynucleoside; and
(f) treating the N-acyl-N-alkyl-O(5′)-DMT deoxynucleoside with a suitable 2-cyanoethyl-N,N-diisopropylaminophosphitylating agent to afford the compound of Formula II.

In a fourth embodiment, the present invention provides for a process for preparing a compound of Formula III:

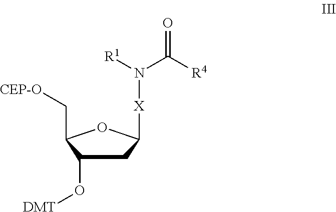

wherein X—N is a heterocyclic nucleobase and its exocyclic amine substituent; R¹ is C₁-C₁₂ n-alkyl; and R⁴ is CH₃, C₂H₅, CF₃, or CCl₃. The process involves the steps of:
(a) treating an N-alkyl deoxynucleoside of the formula

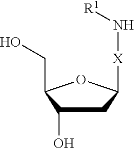

wherein X—N and R¹ are defined as above with a suitable silylating agent and a suitable base to afford an N-alkyl-O(5′)-SPG deoxynucleoside;
(b) treating the N-alkyl-O(5′)-SPG deoxynucleoside with a suitable dimethoxytritylating agent to afford an N-alkyl-O(3′)-DMT-O(5′)-SPG deoxynucleoside;
(c) treating the N-alkyl-O(3′)-DMT-O(5′)-SPG deoxynucleoside with a suitable acylating agent to afford an N-acyl-N-alkyl-O(3′)-DMT-O(5′)-SPG deoxynucleoside;
(d) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(e) treating the N-acyl-N-alkyl-O(3′)-DMT deoxynucleoside with a suitable 2-cyanoethyl-N,N-diisopropylaminophosphitylating agent to afford the compound of Formula III.

In a fifth embodiment, the invention provides for a process for preparing a compound of Formula III:

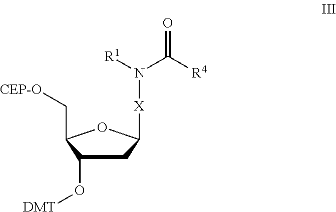

wherein X—N is a heterocyclic nucleobase and its exocyclic amine substituent; R¹ is C₁-C₁₂ n-alkyl; and R⁴ is CH₃, C₂H₅, CF₃, or CCl₃. The process involves the steps of:
(a) treating a deoxynucleoside with a suitable silylating agent and a suitable base to afford an O(5′)-SPG deoxynucleoside;
(b) treating the O(5′)-SPG deoxynucleoside with a suitable dimethoxytritylating agent to afford an O(3′)-DMT-O(5′)-SPG deoxynucleoside;
(c) treating the O(3′)-DMT-O(5′)-SPG deoxynucleoside with a suitable acylating agent to afford an N-acyl-O(3′)-DMT-O(5′)-SPG deoxynucleoside;
(d) treating the N-acyl-O(3′)-DMT-O(5′)-SPG deoxynucleoside with a suitable alkylating agent to afford an N-acyl-N-alkyl-O(3′)-DMT-O(5′)-SPG deoxynucleoside;
(e) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(f) treating the N-acyl-N-alkyl-O(3′)-DMT deoxynucleoside with a suitable 2-cyanoethyl-N,N-diisopropylaminophosphitylating agent to afford the compound of Formula III.

In a sixth embodiment, the present invention provides for a process for preparing a compound of Formula III:

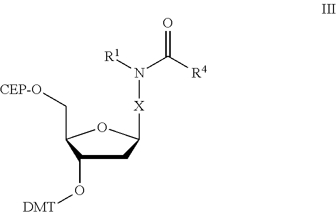

wherein X—N is a heterocyclic nucleobase and its exocyclic amine substituent; R¹ is C₁-C₁₂ n-alkyl; and R⁴ is CH₃, C₂H₅, CF₃, or CCl₃. The process involves the steps of:
(a) treating an N-alkyl deoxynucleoside of the formula

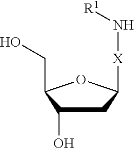

wherein X—N and R¹ are defined as above with a suitable silylating agent and a suitable base to afford an N-alkyl-O(5′)-SPG deoxynucleoside;
(b) treating the N-alkyl-O(5′)-SPG deoxynucleoside with a suitable acylating agent to afford an N-acyl-N-alkyl-O(5′)-SPG deoxynucleoside;
(c) treating the N-acyl-N-alkyl-O(5′)-SPG deoxynucleoside with a suitable dimethoxytritylating agent to afford an N-acyl-N-alkyl-O(3′)-DMT-O(5′)-SPG deoxynucleoside;
(d) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(e) treating the N-acyl-N-alkyl-O(3′)-DMT deoxynucleoside with a suitable 2-cyanoethyl-N,N-diisopropylaminophosphitylating agent to afford the compound of Formula III.

In a seventh embodiment, the present invention provides for a process for preparing a compound of Formula III:

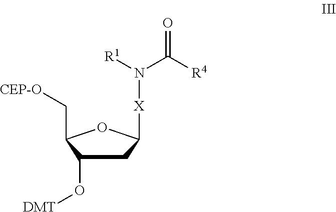

wherein X—N is a heterocyclic nucleobase and its exocyclic amine substituent; R¹ is C₁-C₁₂ n-alkyl; and R⁴ is CH₃, C₂H₅, CF₃, or CCl₃; the process comprising the steps of:
(a) treating a deoxynucleoside with a suitable silylating agent and a suitable base to afford an O(5′)-SPG deoxynucleoside;
(b) treating the O(5′)-SPG deoxynucleoside with a suitable acylating agent to afford an N-acyl-O(5′)-SPG deoxynucleoside;
(c) treating the N-acyl-O(5′)-SPG deoxynucleoside with a suitable dimethoxytritylating agent to afford an N-acyl-O(3′)-DMT-O(5′)-SPG deoxynucleoside;
(d) treating the N-acyl-O(3′)-DMT-O(5′)-SPG deoxynucleoside with a suitable alkylating agent to afford an N-acyl-N-alkyl-O(3′)-DMT-O(5′)-SPG deoxynucleoside;
(e) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(f) treating the N-acyl-N-alkyl-O(3′)-DMT deoxynucleoside with a suitable 2-cyanoethyl-N,N-diisopropylaminophosphitylating agent to afford the compound of Formula III.

### Definitions of Abbreviations

As used herein, the terms and abbreviations set forth below have the following defined meanings.

“Acylating agent” means any reagent that can acylate a primary or secondary amine such as acetic anhydride, acetyl chloride, trichloroacetic anhydride, trichloroacetyl chloride, propionic anhydride, propionyl chloride, trifluoroacetic anhydride, and the like.

“Alkylating agent” means any reagent that can alkylate a primary amine or a secondary amide such as alkyl halides, alkyl sulfates, alkyl sulfonates, and the like.

“Alkyl halide” means a compound of the formula R¹-Hal, wherein R¹ is C₁-C₁₂ n-alkyl and Hal is Cl, Br, or H.

“Alkyl sulfate” means a compound of the formula

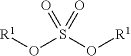

wherein R¹ is C₁-C₁₂ n-alkyl.

“Alkyl sulfonate” means a compound of the formula

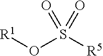

wherein R¹ is C₁-C₁₂ n-alkyl and R⁵ is alkyl or aryl.

“Base” means any organic base typically used in silylation and acylation reactions, such as triethylamine, diisopropylethylamine, imidazole, NMI, pyridine, N,N-dimethylaminopyridine, N-methylmorpholine, collidine, and the like.

“Bis-reagent” means 3-((bis(diisopropylamino)phosphino)oxy)propanenitrile or (i-Pr₂N)₂POCH₂CH₂CN.

“Brine” means saturated aqueous sodium chloride.

“CEP” means 2-cyanoethyloxy-N,N-diisopropylamino-phosphityl or P(OCH₂CH₂CN)N(i-Pr)₂.

“Chlorosilane” means any of TBDMS-Cl, TES-Cl, TIPS-Cl, TBDPS-Cl, and the like.

“CPG” means controlled pore glass, a solid support.

“2-cyanoethyl-N,N-diisopropylaminophosphitylating agent” means any reagent that can donate a CEP group to an alcohol such as bis-reagent, 2-cyanoethyl-N,N-diisopropylaminophosphityl chloride, and the like.

“dA” means 2′-deoxyadenosine.

“dC” means 2′-deoxycytidine.

“dG” means 2′-deoxyguanosine.

“dI” means 2′-deoxyinosine.

“dU” means 2′-deoxyuracil.

“DCM” means dichloromethane.

“Deoxynucleoside” means the repeating synthon of DNA that is composed of a heterocyclic nucleobase and a 2-deoxyribose. As used in this disclosure, deoxynucleoside refers to both natural and unnatural deoxynucleosides that are known by those skilled in the art to be useful to oligonucleotide synthesis.

“Desilylating agent” means any reagent that can remove a silyl protecting group from an SPG-protected alcohol to reveal a free alcohol. Desilylating agents include fluoride sources, such as tetrabutylammonium fluoride, potassium fluoride, hydrogen fluoride, and the like; and acids, such as hydrochloric acid, acetic acid, TFA, toluenesulfonic acid, camphorsulfonic acid, and the like.

“Dimethoxytritylating agent” means a dimethoxytrityl, or bis(4-methoxyphenyl)(phenyl)methyl, electrophile such as DMT-Cl, DMT-OTf, and the like.

“DMF” means N,N-dimethylformamide.

“DMT” means dimethoxytrityl or bis(4-methoxyphenyl)(phenyl)methyl.

“DNA” means (2′-deoxyribo)nucleic acid.

“EtOAc” means ethyl acetate.

“Exocyclic amine substituent” means an amine substituent that is bound to a carbon atom belonging to a carbocylic or heterocyclic ring, wherein the substituent does not itself belong to the ring.

“Heterocyclic nucleobase” means an aromatic structure of one or two rings, containing from 5 to 10 carbon and/or nitrogen ring atoms, and their associated substituents, which are composed of additional hydrogen, carbon, nitrogen, oxygen, sulfur, chlorine, bromine, fluorine and iodine atoms. A heterocyclic nucleobase may be attached to 2-deoxyribose via one of its ring nitrogen atoms to give an N-nucleoside or via one of its ring carbon atoms to give a C-nucleoside.

“MeOH” means methanol.

“n-alkyl” means a compound of formula CₙH₂ₙ₊₁ wherein the carbon atoms are attached in linear fashion. The term “C₁-C₁₂ n-alkyl” refers to a compound of formula CₙH₂ₙ₊₁ containing from 1 to 12 carbon atoms such as methyl, ethyl, n-propyl, n-butyl, n-pentyl, n-hexyl, n-octyl, n-nonyl, n-decyl, n-dodecyl, and the like.

“NMI” means 1-methylimidazole.

“Oligonucleotide” means a segment of single stranded DNA or RNA, typically fewer than 100 nucleotides in length. As used in this disclosure, oligonucleotides may be composed of both natural and unnatural nucleotides and may contain other modifiers and tags that are known in the art to be useful in oligonucleotide synthesis.

“RNA” means ribonucleic acid.

“Silylating agent” means any reagent that can donate an SPG to an alcohol, such as chlorosilanes, silyl sulfonates, and the like.

“Silyl protecting group” or “SPG” means any of TBDMS, TES, TIPDS TIPS, TBDPS, and the like.

“Silyl sulfonate” means a compound of the formula

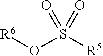

wherein R⁵ is alkyl or aryl and R⁶ is TBDMS, TES, TIPS, TBDPS, or the like.

“TBDMS” means t-butyldimethylsilyl.

“TBDPS” means t-butyldiphenylsilyl.

“TES” means triethylsilyl.

“TFA” means trifluoroacetic acid.

“TIPDS” means 1,1,3,3-tetraisopropyldisiloxane-1,3-diyl, a common silyl protecting group for cyclic protection of both the 3′ and 5′ hydroxyl groups of deoxyribose.

“TIPS” means tri(iso-propyl)silyl.

“THF” means tetrahydrofuran.

### DETAILED DESCRIPTION OF THE INVENTION

The present invention relates to nucleoside derivatives of Formula I. Preferred embodiments of the compounds of Formula I are given below:

(a) Compounds wherein X—N is:
(i) cytosine; or
(ii) adenine;

(b) Compounds wherein R¹ is:
(i) methyl;
(ii) ethyl;
(iii) n-propyl;
(iv) n-butyl;
(v) n-pentyl;
(vi) n-hexyl;
(vii) n-octyl;
(viii) n-nonyl;
(ix) n-decyl; or
(x) n-dodecyl;

(c) Compounds wherein:
(i) R² is CEP and R³ is DMT; or
(ii) R² is DMT and R³ is CEP; and

(d) Compounds wherein R⁴ is:
(i) CH₃;
(ii) C₂H₅;
(iii) CF₃; or
(iv) CCl₃.

It is understood that further preferred embodiments of the compounds of Formula I can be selected by requiring one or more of the preferred embodiments (a) through (d) above of the compounds of Formula I. For example, further preferred embodiments can be obtained by combining (a)(i) and (b)(i); (a)(i) and (b)(ii); (a)(ii) and (b)(i); (a)(ii) and (b)(ii); (a)(i), (b)(i), and (c)(i); (a)(i), (b)(i), and (c)(ii); (a)(i), (b)(ii), and (c)(i); (a)(ii), (b)(i), and (c)(i); (a)(i), (b)(ii), and (c)(ii); (a)(ii), (b)(i), and (c)(ii); (a)(ii), (b)(ii), and (c)(i); (a)(ii), (b)(ii), and (c)(ii); (a)(i), (b)(i), and (d)(i); (a)(i), (b)(i), and (d)(ii); (a)(i), (b)(ii), and (d)(i); (a)(ii), (b)(i), and (d)(i); (a)(i), (b)(ii), and (d)(ii); (a)(ii), (b)(i), and (d)(ii); (a)(ii), (b)(ii), and (d)(i); (a)(ii), (b)(ii), and (d)(ii); (a)(i), (b)(i), and (d)(iii); (a)(i), (b)(i), and (d)(iv); (a)(i), (b)(ii), and (d)(iii); (a)(ii), (b)(i), and (d)(iii); (a)(i), (b)(ii), and (d)(iv); (a)(ii), (b)(i), and (d)(iv); (a)(ii), (b)(ii), and (d)(iii); (a)(ii), (b)(ii), and (d)(iv); (a)(i), (b)(i), (c)(i), and (d)(i); (a)(i), (b)(i), (c)(i), and (d)(ii); (a)(i), (b)(i), (c)(ii), and (d)(i); (a)(i), (b)(ii), (c)(i), and (d)(i); (a)(ii), (b)(i), (c)(i), and (d)(i); (a)(i), (b)(i), (c)(ii), and (d)(ii); (a)(i), (b)(ii), (c)(i), and (d)(ii); (a)(ii), (b)(i), (c)(i), and (d)(ii); (a)(i), (b)(ii), (c)(ii), and (d)(i); (a)(ii), (b)(i), (c)(ii), and (d)(i); (a)(ii), (b)(ii), (c)(i), and (d)(i); (a)(i), (b)(ii), (c)(ii), and (d)(ii); (a)(ii), (b)(i), (c)(ii), and (d)(ii); (a)(ii), (b)(ii), (c)(i), and (d)(ii); (a)(ii), (b)(ii), (c)(ii), and (d)(i); (a)(ii), (b)(ii), (c)(ii), and (d)(ii); (a)(i), (b)(i), (c)(i), and (d)(iii); (a)(i), (b)(i), (c)(i), and (d)(iv); (a)(i), (b)(i), (c)(ii), and (d)(iii); (a)(i), (b)(ii), (c)(i), and (d)(iii); (a)(ii), (b)(i), (c)(i), and (d)(iii); (a)(i), (b)(i), (c)(ii), and (d)(iv); (a)(i), (b)(ii), (c)(i), and (d)(iv); (a)(ii), (b)(i), (c)(i), and (d)(iv); (a)(i), (b)(ii), (c)(ii), and (d)(i); (a)(ii), (b)(i), (c)(ii), and (d)(i); (a)(ii), (b)(ii), (c)(i), and (d)(i); (a)(i), (b)(ii), (c)(ii), and (d)(iv); (a)(ii), (b)(i), (c)(ii), and (d)(iv); (a)(ii), (b)(ii), (c)(i), and (d)(iv); (a)(ii), (b)(ii), (c)(ii), and (d)(i); (a)(ii), (b)(ii), (c)(ii), and (d)(iv); and the like; or by solely requiring (a)(i); (a)(ii); (b)(i); (b)(ii); (b)(iii); (b)(iv); (b)(v); (b)(vi); (b)(vii); (b)(viii); (b)(ix); (b)(x); (c)(i); (c)(ii); (d)(i); (d)(ii); (d)(iii); (d)(iv); and the like.

The present invention also relates to methods of preparing nucleoside derivatives of Formula I. Preferred embodiments of these methods are set forth in Schemes 1-4, below, and in the Examples.

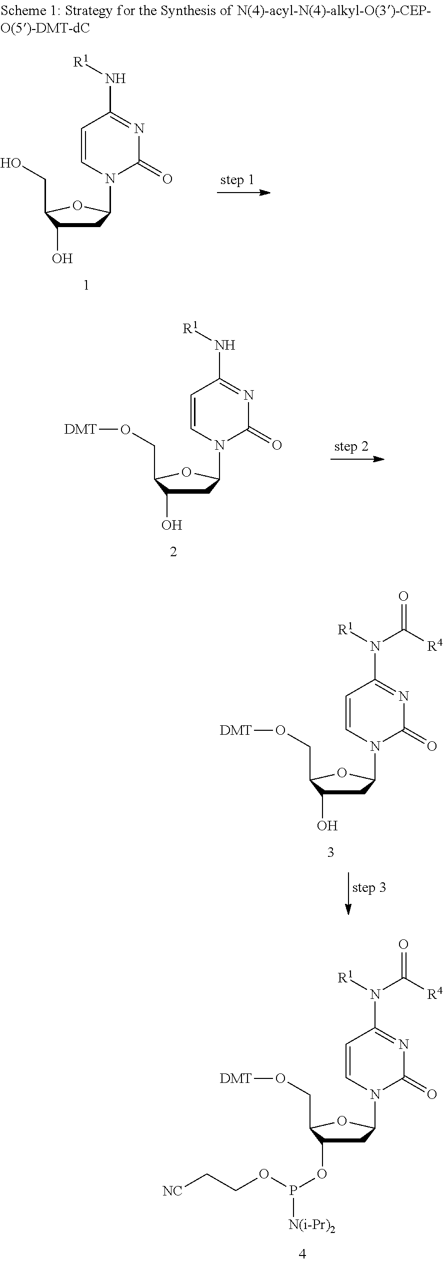

The strategy set forth in Scheme 1 is useful for preparing compounds of Formula I wherein X—N is cytosine, R² is CEP, and R³ is DMT. In step 1, an N(4)-alkyl-dC (1) is converted to an N(4)-alkyl-O(5′)-DMT-dC (2), such as by treatment of 1 with DMT-Cl in anhydrous pyridine. Step 2 involves the N-acylation of 2, such as by acetic anhydride, to afford an N(4)-acyl-N(4)-alkyl-O(5′)-DMT-dC (3). In step 3, 3 is converted to an N(4)-acyl-N(4)-alkyl-O(3′)-CEP-O(5′)-DMT-dC (4), such as by treatment of 3 with bis-reagent and an acid catalyst such as TFA-NMI in anhydrous DCM or by treatment of 3 with 2-cyanoethyl-N,N-diisopropylaminophosphityl chloride and diisopropylethylamine in anhydrous THF.

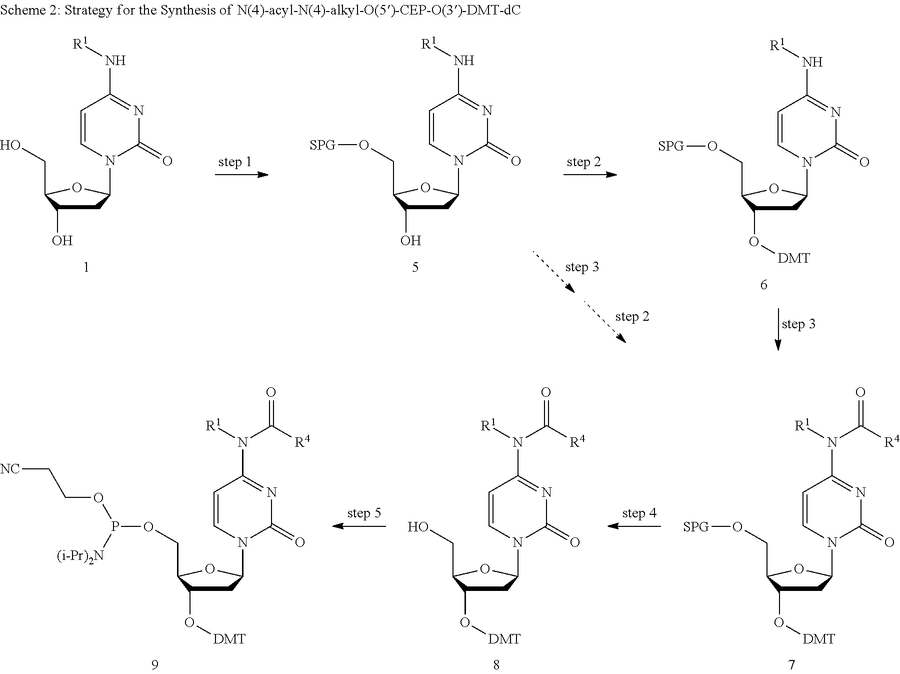

The strategy set forth in Scheme 2 is useful for preparing compounds of Formula I wherein X—N is cytosine, R² is DMT, and R³ is CEP. In step 1, an N(4)-alkyl-dC (1) is converted to an N(4)-alkyl-O(5′)-SPG-dC (5), such as by treatment of 1 with imidazole and one equivalent of a chlorosilane in anhydrous DMF. Step 2 involves the installation of a DMT group at the O(3′)-position of 5 to afford an N(4)-alkyl-O(3′)-DMT-O(5′)-SPG-dC (6), such as by treatment of 5 with DMT-Cl in anhydrous pyridine. In step 3, 6 is converted to an N(4)-acyl-N(4)-alkyl-O(3′)-DMT-O(5′)-SPG-dC (7) by treatment of 6 with an acylating reagent, such as acetic anhydride or acetyl chloride. In some cases, step 3 may be performed before step 2. Step 4 involves the removal of the O(5′)-silyl protecting group from 7 to afford an N(4)-acyl-N(4)-alkyl-O(3′)-DMT-dC (8), such as by treatment of 7 with tetrabutylammonium fluoride in anhydrous THF. In step 5, 8 is converted to an N(4)-acyl-N(4)-alkyl-O(5′)-CEP-O(3′)-DMT-dC (9), such as by the treatment of 8 with bis-reagent and an acid catalyst such as TFA-NMI in anhydrous DCM or by treatment of 8 with 2-cyanoethyl-N,N-diisopropylaminophosphityl chloride and diisopropylethylamine in anhydrous THF.

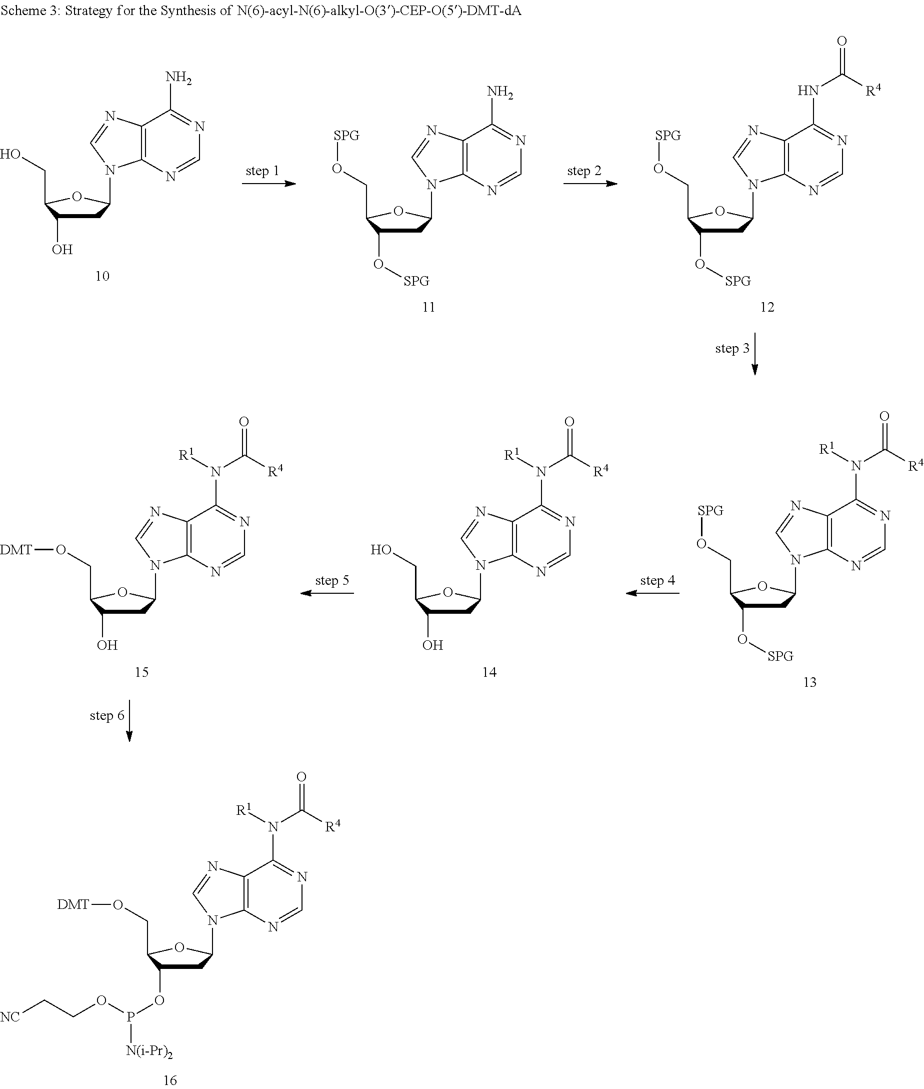

The strategy set forth in Scheme 3 is useful for preparing compounds of Formula I wherein X—N is adenine, R² is CEP, and R³ is DMT. In step 1, dA (10) is converted to an O(3′)-O(5′)-bis(SPG)-dA (11), such as by treatment of 10 with imidazole and two equivalents of a chlorosilane in anhydrous DMF. Step 2 involves the N-acylation of 11, such as by acetyl chloride in anhydrous pyridine, to afford an N(6)-acyl-O(3′)-O(5′)-bis(SPG)-dA (12). In step 3, 12 is converted to an N(6)-acyl-N(6)-alkyl-O(3′)-O(5′)-bis(SPG)-dA (13), such as by treatment of 12 with an alkyl halide in DCM. Step 4 involves the bis-deprotection of 13 to afford an N(6)-acyl-N(6)-alkyl-dA (14), such as by treatment of 13 with tetrabutylammonium fluoride in anhydrous THF. In step 5, 14 is converted to an N(6)-acyl-N(6)-alkyl-O(5′)-DMT-dA (15), such as by the treatment of 14 with DMT-Cl in anhydrous pyridine. Step 6 involves the O(5′)-protection of 15 to afford an N(6)-acyl-N(6)-alkyl-O(3′)-CEP-O(5′)-DMT-dA (16), such as by the treatment of 15 with bis-reagent and an acid catalyst such as TFA-NMI in anhydrous DCM or by treatment of 15 with 2-cyanoethyl-N,N-diisopropylaminophosphityl chloride and diisopropylethylamine in anhydrous THF.

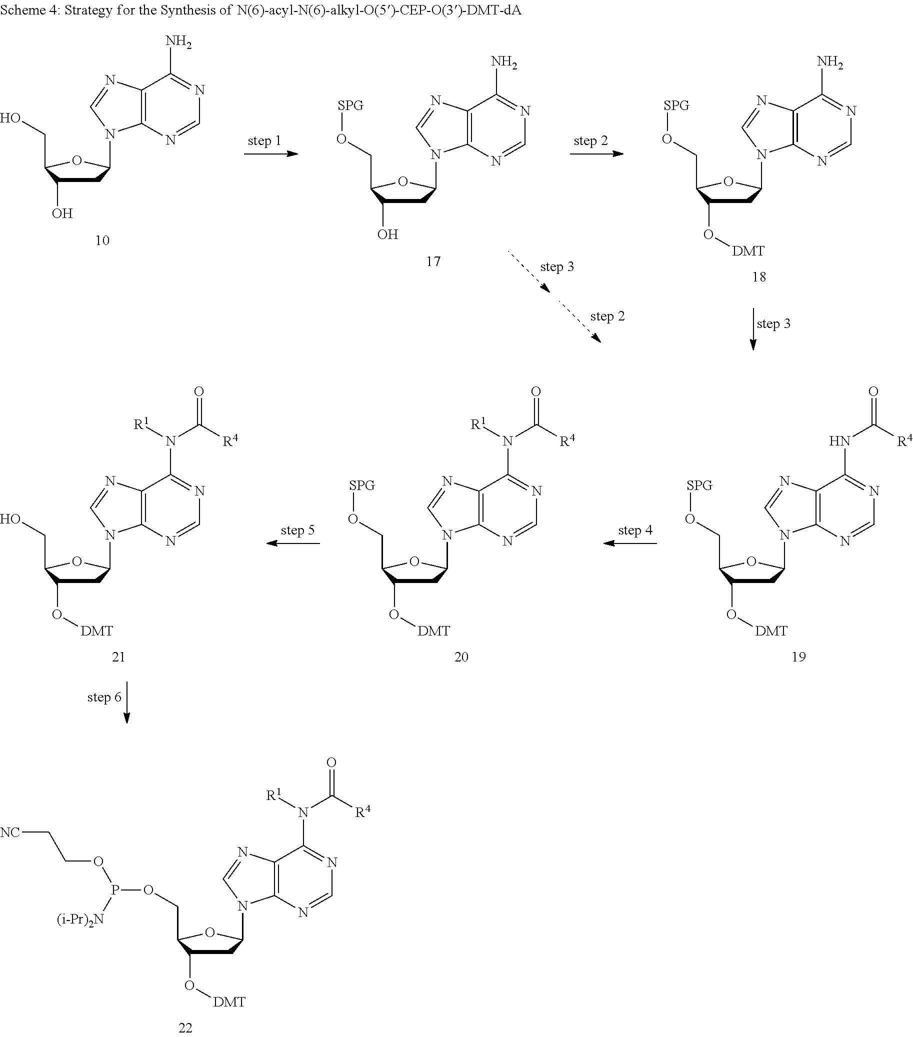

The strategy set forth in Scheme 4 is useful for preparing compounds of Formula I wherein X—N is adenine, R² is DMT, and R³ is CEP. In step 1, dA (10) is converted to an O(5′)-SPG-dA (17), such as such as by treatment of 10 with imidazole and one equivalent of a chlorosilane in anhydrous DMF. Step 2 involves the installation of a DMT group at the O(3′)-position of 17 to afford an O(3′)-DMT-O(5′)-SPG-dC (18), such as by treatment of 17 with DMT-Cl in anhydrous pyridine. In step 3, 18 is converted to an N(6)-acyl-O(3′)-DMT-O(5′)-SPG-dC (19) by treatment of 18 with an acylating reagent, such as acetic anhydride or acetyl chloride. In some cases, step 3 may be performed before step 2. Step 4 involves the N-alkylation of 19, such as by the treatment of 19 with an alkyl halide in DCM, to afford an N(6)-acyl-N(6)-alkyl-O(3′)-DMT-O(5′)-SPG-dC (20). In step 5, removal of the O(5′)-silyl protecting group from 20 affords and N(6)-acyl-N(6)-alkyl-O(3′)-DMT-dC (21). This transformation may be accomplished by the treatment of 20 with tetrabutylammonium fluoride in anhydrous THF. Step 6 involves the O(5′)-protection of 21 to afford an (6)-acyl-N(6)-alkyl-O(5′)-CEP-O(3′)-DMT-dA (22), such as by the treatment of 15 with bis-reagent and an acid catalyst such as TFA-NMI in anhydrous DCM or by treatment of 15 with 2-cyanoethyl-N,N-diisopropylaminophosphityl chloride and diisopropylethylamine in anhydrous THF.

Some variation of Schemes 1-4 may be required for certain compounds of Formula I. It is within the realm of expertise of those skilled in the art of organic synthesis to add protection and deprotection steps, to rearrange the order of steps, and/or to adjust to conditions of various steps in order to accommodate specific compounds of Formula I that are not optimally produced by the routes shown in Schemes 1-4.

The N-acyl-N-alkyl deoxynucleoside derivatives of Formula I, synthesized in the manner set forth in Schemes 1-4, may be employed in automated DNA synthesis to introduce N-alkyl deoxynucleosides into DNA oligonucleotides (Schemes 5 and 6). As will be appreciated by those skilled in the art of DNA synthesis, such automated synthesis may be conducted as follows: (1) a desired DNA sequence is programmed into an automated DNA synthesizer that has been equipped with the necessary reagents; (2) the synthesizer carries out the synthesis steps in automated fashion over a number of cycles, adding each prescribed nucleotide of the sequence until the full-length, protected oligonucleotide is prepared on a CPG support; and (3) the oligonucleotide is cleaved from the CPG support, and protecting groups are removed, to give the free oligonucleotide.

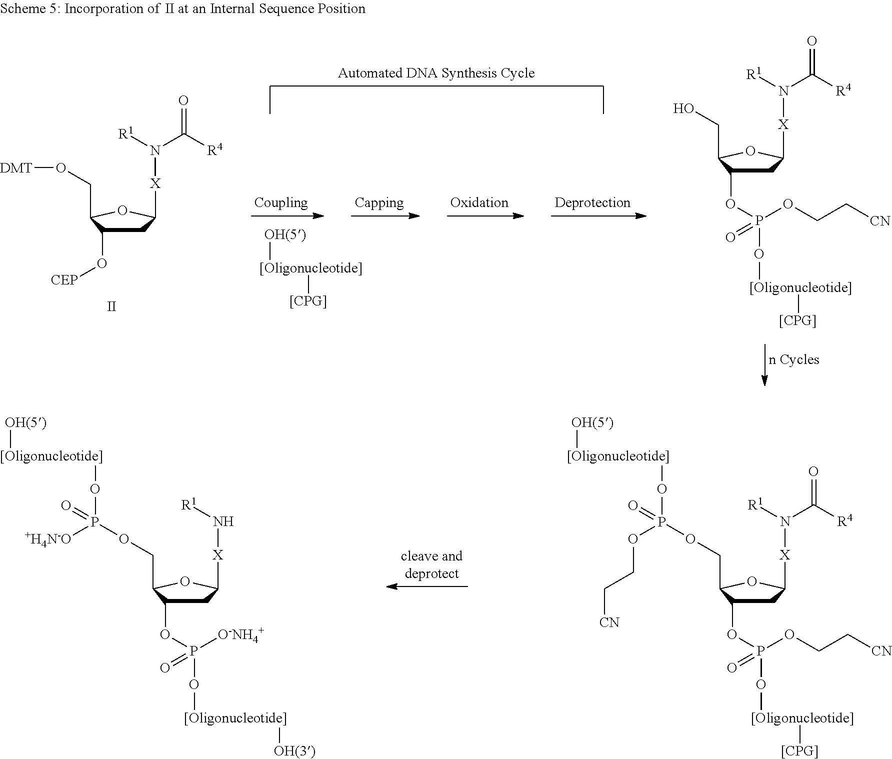

Scheme 5 illustrates a method for the introduction of an N-acyl-N-alkyl-O(3′)-CEP-O(5′)-DMT deoxynucleoside (Formula II) into a DNA oligonucleotide at an internal sequence position. The synthesis of the oligonucleotide is conducted in the 3′ to 5′ direction. Using a similar technique, it is also possible to incorporate a compound of Formula II at the 5′-terminus of the oligonucleotide.

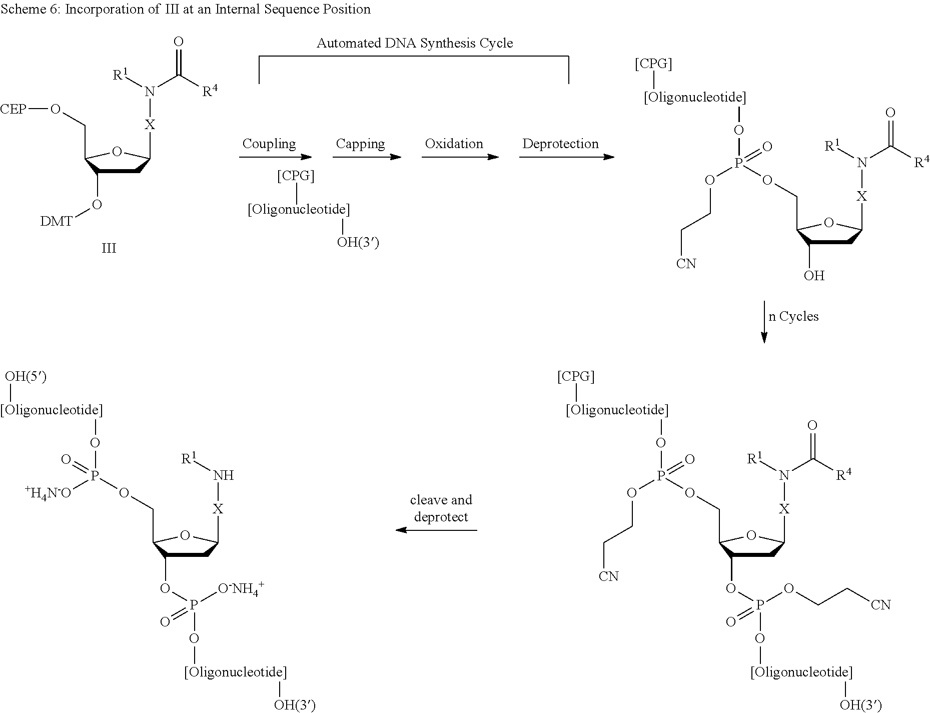

Scheme 6 illustrates a method for the introduction of an N-acyl-N-alkyl-O(5′)-CEP-O(3′)-DMT deoxynucleoside (Formula III) into a DNA oligonucleotide at an internal sequence position. The synthesis of the oligonucleotide is conducted in the 5′ to 3′ direction. Using a similar technique, it is also possible to incorporate a compound of Formula III at the 3′-terminus of the oligonucleotide.

### EXAMPLE 1

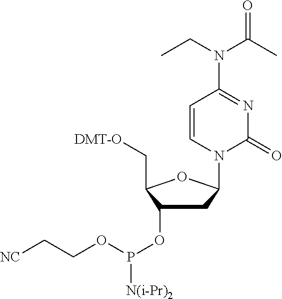

### N(4)-Acetyl-O(3′)-CEP-O(5)-DMT-N(4)-ethyl-dC (4a)

N(4)-Acetyl-O(3′)-CEP-O(5′)-DMT-N(4)-ethyl-dC (4a) was prepared according to the strategy set forth in Scheme 1.

Step 1: Synthesis of O(5′)-DMT-N(4)-ethyl-dC (2a). A solution of N(4)-ethyl-dC (1a, 1.0 g) in anhydrous pyridine (24 mL) was treated with a solution of DMT-Cl (1.6 g) in anhydrous pyridine (10 mL). The resulting mixture was stirred overnight at room temperature under a dry atmosphere. MeOH (1 mL) was added and the reaction mixture was concentrated in vacuo at 30-35° C. The residue was partitioned between DCM and saturated aqueous sodium bicarbonate (25 mL each). The layers were separated, and the organic layer was dried over anhydrous sodium sulfate, filtered, and concentrated at reduced pressure. The product was purified by flash chromatography on silica gel, eluting with a gradient of 4-10% MeOH in DCM. Fractions containing pure product were combined and concentrated at reduced pressure. The residue was further dried in vacuo overnight to afford 2a. TLC (silica gel on glass, 90DCM:10MeOH) shows a single spot, Rf=0.45. MS (AP+): 558 (m+1).

Step 2: Synthesis of N(4)-acetyl-O(5′)-DMT-N(4)-ethyl-dC (3a). A solution of 2a prepared in Step 1 (1.0 g) in anhydrous DMF (10 mL) was placed under a dry atmosphere and cooled in an ice water bath for 15 minutes. Acetic anhydride (1.7 mL) was added drop-wise over 1 minute. After another 30 minutes the cold bath was removed and the reaction was stirred at room temperature for 24 hours. The reaction mixture was concentrated in vacuo. The resulting residue was partitioned between EtOAc and saturated aqueous sodium bicarbonate (100 mL each). The layers were separated, and the organic layer was dried over anhydrous sodium sulfate, filtered, and concentrated at reduced pressure. The product was purified by flash chromatography on silica gel, eluting with a gradient of 50-100% EtOAc in hexane. Fractions containing pure product were combined and concentrated at reduced pressure. The residue was re-dissolved in DCM and evaporated to a solid which was further dried in vacuo overnight to afford 3a. TLC (silica gel on glass, 90EtOAc:10MeOH) shows a single spot, Rf=0.58. MS (AP+): 600 (m+1).

Step 3: Synthesis of N(4)-Acetyl-O(3′)-CEP-O(5′)-DMT-N(4)-ethyl-dC (4a). A solution of 3a prepared in Step 2 (0.82 g) in anhydrous DCM (20 mL) was placed under a dry atmosphere and treated with bis-reagent (0.6 mL). The resulting mixture was stirred at room temperature for 5 minutes, and then a solution consisting of 0.5M NMI and 0.25M TFA in DCM (2.7 mL) was added. The resulting mixture was stirred at room temperature for 3.5 hours then diluted with DCM (30 mL). The diluted solution was sequentially washed with saturated aqueous sodium bicarbonate (30 mL), water (2×30 mL), and saturated aqueous sodium chloride (30 mL). The organic layer was dried over anhydrous sodium sulfate, filtered, and concentrated at reduced pressure. Silica gel (30 g) was slurried in 50EtOAc:49hexane:1Et₃N and packed into a 4 cm diameter column. The crude product was purified on this column, eluting with a gradient of 50-80%EtOAc in hexane, buffered by 1% Et₃N. Fractions containing pure product were combined and concentrated at reduced pressure. The residue was re-dissolved in anhydrous MeCN, filtered to remove haziness, and evaporated at reduced pressure. The purified product was re-dissolved in anhydrous DCM and evaporated at reduced pressure to a crisp foam which was further dried in vacuo overnight to afford 4a. ³¹P-NMR (CD₃CN) shows a single resonance at 148.73 ppm. MS (AP+): 800 (m+1), 822 (m+Na), 839 (m+K).

### EXAMPLE 2

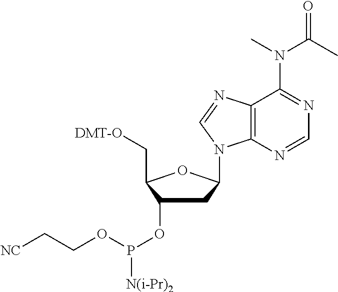

### N(6)-Acetyl-O(3)-CEP-O(5)-DMT-N(6)-methyl-dA (16a)

N(6)-Acetyl-O(3′)-CEP-O(5′)-DMT-N(6)-methyl-dA (16a) was prepared according to the strategy set forth in Scheme 3.

Step 1: Synthesis of O(3′),O(5′)-bis(TBDMS)-dA (11a). An anhydrous mixture of dA (10, 10 g), imidazole (4.1 g), and DMF (150 mL) was placed under a dry atmosphere and cooled in an ice/water bath for 15 minutes. A solution of TBDMS-Cl (9 g) in anhydrous THF (25 mL) was added over 5 minutes. The resulting mixture was stirred at room temperature for 24 hours. The solution was concentrated in vacuo and the residual syrup was partitioned between EtOAc (300 mL), Hexane (30 mL), and water (300 mL). The layers were separated, and the organic layer was washed with 10% aqueous sodium bicarbonate (2×300 mL), dried over dried over anhydrous sodium sulfate, filtered, and concentrated at reduced pressure. The solid was triturated with hexanes, filtered, and dried in vacuo to afford 11a. TLC (silica gel on glass, 50EtOAc:50DCM) shows a single spot, Rf=0.30.

Step 2: Synthesis of N(6)-acetyl-O(3′),O(5′)-bis(TBDMS)-dA (12a). A solution of 11a prepared in Step 1 (13.8 g) in anhydrous pyridine (160 mL) was placed under a dry atmosphere and cooled in an ice water bath for 15 minutes. Acetyl chloride (2.7 mL) was added dropwise. The reaction mixture was stirred under a dry atmosphere for 5 hours, gradually warming to room temperature. Concentrated aqueous ammonia (1.0 mL) was added and the reaction mixture was stirred for 5 minutes at room temperature before being concentrated in vacuo. The residue was partitioned between EtOAc and brine (200 mL each). The layers were separated, and the organic layer was dried over anhydrous sulfate, filtered, and concentrated at reduced pressure. The product was purified by flash chromatography on silica gel, eluting with 75DCM:25EtOAc. Fractions containing pure product were combined and evaporated at reduced pressure. The solid product was further dried in vacuo overnight to afford 12a. TLC (silica gel on glass, 50EtOAc:50DCM) shows a single spot, Rf=0.62. MS (AP+): 522 (m+1).

Step 3: Synthesis of N(6)-acetyl-O(3′),O(5′)-bis(TBDMS)-N(6)-methyl-dA (13a). A solution of 12a prepared in Step 2 (15.0 g) in DCM (280 mL) was treated with tetrabutylammonium bromide, 1N aqueous sodium hydroxide (280 mL), and methyl iodide (7.2 mL). The resulting two-phase mixture was stirred vigorously at room temperature for 15 minutes. The layers were separated, and the organic layer was washed with water (250 mL). The organic layer was then dried over anhydrous sodium sulfate, filtered, and concentrated at reduced pressure. The product was purified by flash chromatography on silica gel, eluting with 89DCM:11EtOAc. Fractions containing pure product were combined and evaporated at reduced pressure. The solid product was further dried in vacuo overnight to afford 13a. TLC (silica gel on glass, 50EtOAc:50DCM) shows a single spot, Rf=0.48. MS (AP+): 536 (m+1).

Step 4: Synthesis of N(6)-acetyl-N(6)-methyl-dA (14a). A solution of 13a prepared in Step 3 (15.0 g) in anhydrous THF (100 mL) was placed under a dry atmosphere and treated with an anhydrous 1M solution of tetrabutylammonium fluoride in THF (84 mL). As soon as the reaction was judged complete by TLC (silica gel on glass, 93EtOAc:7MeOH), the reaction solution was concentrated to 50 mL at reduced pressure. The concentrate was applied to a column of silica gel (200 g) in EtOAc. The column was eluted with EtOAc (1 L) followed by 96EtOAc:4MeOH (6 L). Fractions containing pure product were combined and evaporated at reduced pressure. The solid product was triturated with diethyl ether and collected by filtration. It was then further dried in vacuo overnight to afford 14a. TLC (silica gel on glass, 85EtOAc:15 MeOH) shows a single spot, Rf=0.72. MS (AP+): 308 (m+1).

Step 5: Synthesis of N(6)-acetyl-O(5′)-DMT-N(6)-methyl-dA (15a). A solution of 14a prepared in Step 4 (5.9 g) in anhydrous pyridine (200 mL) was placed under a dry atmosphere and cooled on an ice bath for 15 minutes. Solid DMT-Cl (7.0 g) was added and the reaction mixture was stirred at room temperature for 6 hours. The solution was concentrated in vacuo, keeping the temperature below 35° C. The residue was partitioned between EtOAc (200 mL), water (100 mL), and brine (100 mL). The layers were separated, and the organic layer was dried over anhydrous sodium sulfate, filtered, and concentrated at reduced pressure. The product was purified by flash chromatography on silica gel, eluting with a gradient of 50-100% EtOAc in hexane. Fractions containing pure product were combined and evaporated at reduced pressure to a crisp foam. The product was then further dried in vacuo overnight to afford 15a. TLC (silica gel on glass, EtOAc) shows a single spot, Rf=0.50. MS (AP+): 608 (m+1).

Step 6: Synthesis of N(6)-acetyl-O(3′)-CEP-O(5′)-DMT-N(6)-methyl-dA (16a). A solution of 15a prepared in Step 5 (0.85 g) in anhydrous DCM (20 mL) was placed under a dry atmosphere and treated with bis-reagent (0.65 mL). The resulting mixture was stirred at room temperature for 5 minutes, and then a solution consisting of 0.5M NMI and 0.25M TFA in DCM (3 mL) was added. The resulting solution was stirred at room temperature for 4 hours and was then diluted with DCM (30 mL). The diluted solution was sequentially washed with water (2×30 mL), and saturated aqueous sodium bicarbonate (30 mL). The organic layer was dried over anhydrous sodium sulfate, filtered, and concentrated at reduced pressure. The resulting residue was dissolved in DCM (8 mL) and added drop-wise to vigorously stirred pentane (250 mL). After allowing the solid to settle, the pentane was decanted and the solid was rinsed with additional pentane (40 mL). The remaining solid was dissolved in acetonitrile, and the resulting solution was dried over anhydrous sodium sulfate, filtered, and concentrated at reduced pressure. The residue was re-dissolved in anhydrous DCM and evaporated to a crisp foam at reduced pressure. The purified product was further dried in vacuo overnight to afford 16a. MS (AP+): 808 (m+1), 830 (m+Na).

What is claimed is:

1. A compound of Formula I: 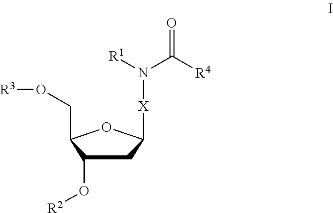
wherein:
X—N is a heterocyclic nucleobase and its exocyclic amine substituent;
R¹ is C₁-C₁₂ n-alkyl;
R² and R³ are each independently CEP or DMT; and
R⁴ is CH₃, C₂H₅, CF₃, or CCl₃;
with the proviso that R² and R³ are different.

2. The compound of claim 1, wherein X—N is cytosine and its exocyclic amine substituent.

3. The compound of claim 2, wherein R¹ is ethyl, R² is CEP, R³ is DMT, and R⁴ is CH₃.

4. The compound of claim 2, wherein R¹ is ethyl, R² is DMT, R³ is CEP, and R⁴ is CH₃.

5. The compound of claim 1, wherein X—N is adenine and its exocyclic amine substituent.

6. The compound of claim 5, wherein R¹ is methyl, R² is CEP, R³ is DMT, and R⁴ is CH₃.

7. The compound of claim 5, wherein R¹ is methyl, R² is DMT, R³ is CEP, and R⁴ is CH₃.

8. A process for preparing a compound of Formula II: 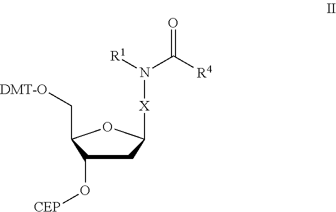
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating an N-alkyl deoxynucleoside of the formula 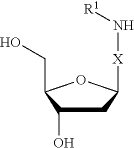
wherein X—N and R¹ are defined as above with a suitable dimethoxytritylating agent to afford an N-alkyl-O(5′)-DMT deoxynucleoside;
(b) treating the N-alkyl-O(5′)-DMT deoxynucleoside with a suitable acylating agent to afford an N-acyl-N-alkyl-O(5′)-DMT deoxynucleoside; and
(c) treating the N-acyl-N-alkyl-O(5′)-DMT deoxynucleoside with a suitable 2-cyanoethyl-N,N-diisopropylaminophosphitylating agent to afford the compound of Formula II.

9. The process of claim 8, wherein the N-alkyl deoxynucleoside is an N(4)-alkyl-dC.

10. A process for preparing a compound of Formula II: 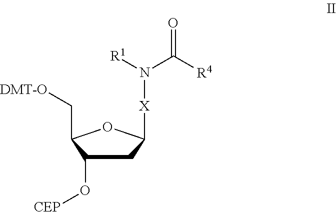
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating a deoxynucleoside with a suitable silylating agent and a suitable base to afford an O(3′)-SPG-O(5′)-SPG deoxynucleoside;
(b) treating the O(3′)-SPG-O(5′)-SPG deoxynucleoside with a suitable acylating agent to afford an N-acyl-O(3′)-SPG-O(5′)-SPG deoxynucleoside;
(c) treating the N-acyl-O(3′)-SPG-O(5′)-SPG deoxynucleoside with a suitable alkylating agent to afford an N-acyl-N-alkyl-O(3′)-SPG-O(5′)-SPG deoxynucleoside;
(d) desilylating the N-acyl-N-alkyl-O(3′)-SPG-O(5′)-SPG deoxynucleoside by treating the N-acyl-N-alkyl-O(3′)-SPG-O(5′)-SPG deoxynucleoside with a suitable desilylating agent to afford an N-acyl-N-alkyl deoxynucleoside;
(e) treating the N-acyl-N-alkyl deoxynucleoside with a suitable dimethoxytritylating agent to afford an N-acyl-N-alkyl-O(5′)-DMT deoxynucleoside; and
(f) treating the N-acyl-N-alkyl-O(5′)-DMT deoxynucleoside with a suitable 2-cyanoethyl-N,N-diisopropylaminophosphitylating agent to afford the compound of Formula II.

11. The process of claim 10, wherein the deoxynucleoside is dA.

12. A process for preparing a compound of Formula III: 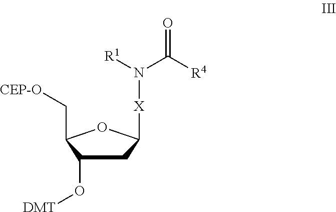
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating an N-alkyl deoxynucleoside of the formula 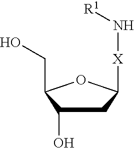
wherein X—N and R¹ are defined as above with a suitable silylating agent and a suitable base to afford an N-alkyl-O(5′)-SPG deoxynucleoside;
(b) treating the N-alkyl-O(5′)-SPG deoxynucleoside with a suitable dimethoxytritylating agent to afford an N-alkyl-O(3′)-DMT-O(5′)-SPG deoxynucleoside;
(c) treating the N-alkyl-O(3′)-DMT-O(5′)-SPG deoxynucleoside with a suitable acylating agent to afford an N-acyl-N-alkyl-O(3′)-DMT-O(5′)-SPG deoxynucleoside;
(d) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(e) treating the N-acyl-N-alkyl-O(3′)-DMT deoxynucleoside with a suitable 2-cyanoethyl-N,N-diisopropylaminophosphitylating agent to afford the compound of Formula III.

13. The process of claim 12, wherein the N-alkyl deoxynucleoside is an N(4)-alkyl-dC.

14. A process for preparing a compound of Formula III: 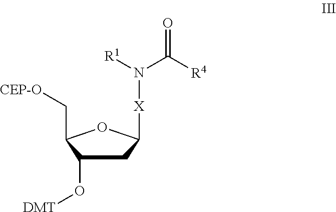
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating a deoxynucleoside with a suitable silylating agent and a suitable base to afford an O(5′)-SPG deoxynucleoside;
(b) treating the O(5′)-SPG deoxynucleoside with a suitable dimethoxytritylating agent to afford an O(3′)-DMT-O(5′)-SPG deoxynucleoside;
(c) treating the O(3′)-DMT-O(5′)-SPG deoxynucleoside with a suitable acylating agent to afford an N-acyl-O(3′)-DMT-O(5′)-SPG deoxynucleoside;
(d) treating the N-acyl-O(3′)-DMT-O(5′)-SPG deoxynucleoside with a suitable alkylating agent to afford an N-acyl-N-alkyl-O(3′)-DMT-O(5′)-SPG deoxynucleoside;
(e) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(f) treating the N-acyl-N-alkyl-O(3′)-DMT deoxynucleoside with a suitable 2-cyanoethyl-N,N-diisopropylaminophosphitylating agent to afford the compound of Formula III.

15. The process of claim 14, wherein the deoxynucleoside is dA.

16. A process for preparing a compound of Formula III: 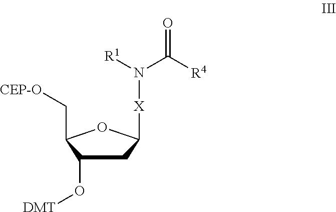
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating an N-alkyl deoxynucleoside of the formula 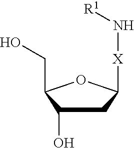
wherein X—N and R¹ are defined as above with a suitable silylating agent and a suitable base to afford an N-alkyl-O(5′)-SPG deoxynucleoside;
(b) treating the N-alkyl-O(5′)-SPG deoxynucleoside with a suitable acylating agent to afford an N-acyl-N-alkyl-O(5′)-SPG deoxynucleoside;
(c) treating the N-acyl-N-alkyl-O(5′)-SPG deoxynucleoside with a suitable dimethoxytritylating agent to afford an N-acyl-N-alkyl-O(3′)-DMT-O(5′)-SPG deoxynucleoside;
(d) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(e) treating the N-acyl-N-alkyl-O(3′)-DMT deoxynucleoside with a suitable 2-cyanoethyl-N,N-diisopropylaminophosphitylating agent to afford the compound of Formula III.

17. The process of claim 16, wherein the N-alkyl deoxynucleoside is an N(4)-alkyl-dC.

18. A process for preparing a compound of Formula III: 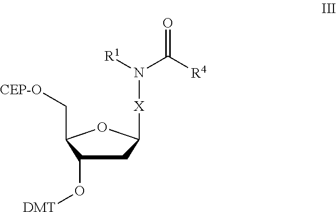
wherein:
X—N is a heterocyclic nucleobase and its exocyclic amine substituent;
R¹ is C₁-C₁₂ n-alkyl; and
R⁴ is CH₃, C₂H₅, CF₃, or CCl₃; the process comprising the steps of:
(a) treating a deoxynucleoside with a suitable silylating agent and a suitable base to afford an O(5′)-SPG deoxynucleoside;
(b) treating the O(5′)-SPG deoxynucleoside with a suitable acylating agent to afford an N-acyl-O(5′)-silyl deoxynucleoside;
(c) treating the N-acyl-O(5′)-SPG deoxynucleoside with a suitable dimethoxytritylating agent to afford an N-acyl-O(3′)-DMT-O(5′)-SPG deoxynucleoside;
(d) treating the N-acyl-O(3′)-DMT-O(5′)-SPG deoxynucleoside with a suitable alkylating agent to afford an N-acyl-N-alkyl-O(3′)-DMT-O(5′)-SPG deoxynucleoside;
(e) desilylating the N-acyl-N-alkyl-O(3′)-DMT-O(5′)-SPG deoxynucleoside by treating the N-acyl-N-alkyl-O(3′)-DMT-O(5′)-SPG deoxynucleoside with a suitable desilylating agent to afford an N-acyl-N-alkyl-O(3′)-DMT deoxynucleoside; and
(f) treating the N-acyl-N-alkyl-O(3′)-DMT deoxynucleoside with a suitable 2-cyanoethyl-N,N-diisopropylaminophosphitylating agent to afford the compound of Formula III.

19. The process of claim 18, wherein the deoxynucleoside is dA.
